(19) United States
(12) Patent Application Publication
(10) Pub. No.: US 2012/0035498 A1
(43) Pub. Date: Feb. 9, 2012

(54) APPARATUS AND METHOD FOR IMPROVING EYE-HAND COORDINATION

(76) Inventor: Larry C. Wilkins, Ft. Lauderdale, FL (US)

(21) Appl. No.: 12/849,874

(22) Filed: Aug. 4, 2010

(51) Int. Cl. A61B 5/103

(52) U.S. Cl. 600/558

(57) ABSTRACT

An apparatus for measuring and quantifying eye-hand coordination of a subject user. The apparatus includes a processor, a tablet interfaced with the processor and that is configured to accept the progressive input of the subject user, and a memory interfaced to the processor that maintains one or more records associated with eye-hand coordination testing of the subject user. The apparatus is configured to progressively display a visual symbol on the tablet and detect a progressive tracing of the displayed visual symbol based upon the input from the subject user. The apparatus is further configured to determine a score based upon at least one characteristic of the progressive tracing, such as how quickly the tracing follows the progressive display or how far the tracing deviates from the path of the progressive display, and then stores the determined score within a record in memory.

### FIELD OF THE DISCLOSURE

The present disclosure relates to apparatus and methods for improving eye-hand coordination. In particular, the present disclosure relates to an apparatus for measuring and quantifying eye-hand coordination using progressive displaying and tracing techniques and related methods.

### BACKGROUND

Eye-hand coordination is the coordinated movement of a subject user's eye as the user's brain processes visual stimuli with the movement of the user's hand. In other words, it is the ability of the subject user's vision processing system to coordinate information received through the eyes to control and guide movement of the subject user's hands.

Eye-hand coordination is important for many day-to-day activities, such as writing, driving, or operating a computer. Beyond such basic needs, eye-hand coordination measurement and quantification is important to understand for particular individuals, such as athletes where activities may include catching a ball or making coordinated movements of the hands relative to a sports object (e.g., a baseball bat as a baseball approaches the subject user or a tennis racquet as a tennis ball moves towards the subject user).

Hand-eye coordination problems are usually first noted in children as a lack of skill in drawing or writing. For impaired children, drawing may show poor orientation on the page and the child may be unable to stay “within the lines” when using a coloring book. The child may continue to depend on his or her hand for inspection and exploration of toys or other objects.

Poor hand-eye coordination can have a wide variety of causes. Some common conditions responsible for inadequate eye-hand coordination include aging, vision problems and movement disorders. More specifically, impairments to eye-hand coordination are known to occur due to brain damage, degeneration of the brain, or other clinical conditions or problems. Adults having Parkinson's disease have a tendency to have increasing difficulty with eye-hand coordination as the disease progresses over time. Other movement disorders exhibiting eye-hand coordination issues include hypertonia (a condition characterized by an abnormal increase in muscle tension and a decreased ability of the muscle to stretch) and ataxia (a condition characterized by a lack of coordination while performing voluntary movements).

In order to treat such impairments, it is desired to repeatedly and consistently measure and quantify the eye-hand coordination of a subject user. Accordingly, there is a need to improve how to measure and quantify eye-hand coordination that permits individualized scoring from different visual stimuli presented to a subject user. Such improved methods and systems for measuring and quantifying eye-hand coordination may be used by insurance companies, which may desire quantifiable tests that analyze a subject user's eye-hand coordination and historically track the subject user's improvement over time. Thus, it may be desirable to provide an apparatus and/or related methods for improving eye-hand coordination that permits improved measuring and quantification of improvements to eye-hand coordination over time.

### SUMMARY

In the following description, certain aspects and embodiments will become evident. It should be understood that the aspects and embodiments, in their broadest sense, could be practiced without having one or more features of these aspects and embodiments. Thus, it should be understood that these aspects and embodiments are merely exemplary.

One aspect of the disclosure relates to an apparatus for measuring and quantifying eye-hand coordination of a subject user. The apparatus may include a processor, a tablet, and a memory. The table is interfaced with the processor and configured to accept progressive input from the subject user. The memory is interfaced with the processor and configured to maintain a record associated with measuring the eye-hand coordination of the subject. The processor is configured to progressively display a visual symbol on the tablet, detect a progressive tracing of the displayed visual symbol from the progressive input of the subject user, determine a score based upon a characteristic of the progressive tracing, and store the determined score within the record in the memory.

Another aspect of the disclosure relates to an apparatus for measuring and quantifying eye-hand coordination of a subject user, where the apparatus may include a housing, a processor disposed within the housing, a tablet, a stylus, a measurement result interface, and a memory. The housing has a first display opening and a second display opening. The tablet is in communication with the processor and disposed within the housing such that a display surface of the tablet is oriented for viewing through the first display opening of the housing. The tablet is configured to accept progressive input from the subject user, who is operating the stylus. The tablet is configured to detect the presence of the stylus as it moved relative to the display surface of the table by the subject user over time as the progressive input of the subject user. The measurement result interface is in communication with the processor and disposed within the housing such that the measurement result interface is viewable through the second display opening of the housing. The memory is in communication with the processor and configured to maintain a plurality of visual cues and a plurality of records associated with measuring the eye-hand coordination of the subject user. As part of this apparatus, the processor is configured to select one of a plurality of visual cues stored in the memory as a visual symbol to be progressively displayed for the subject user based upon an analysis of previously determined eye-hand coordination scores for the subject user stored within the records in the memory, progressively display the selected visual symbol on the tablet, detect a progressive tracing of the displayed visual symbol from the progressive input of the subject user, determine a new eye-hand coordination score based upon how quickly the progressive tracing follows the progressive display of the visual symbol over time and how far a path of the progressive tracing deviates from a path of the progressive display of the visual symbol over time, and store the new eye-hand coordination score within a new record in the memory. The processor may be configured to progressively remove an older portion of the visual symbol while progressively displaying a newer part of the visual symbol.

The apparatus, according to this aspect of the disclosure, may also have the processor being configured to provide a ranking on measurement result interface of the new eye-hand coordination score for the subject user in comparison to at least one prior score for the subject user stored within the records in the memory so as to quantify an improvement factor of the eye-hand coordination of the subject user over time.

Yet a further aspect of the disclosure relates to a method for measuring and quantifying eye-hand coordination of a subject user. The method begins by progressively displaying a visual symbol on a tablet and accepting progressive input from a stylus operated by the subject user as a visual symbol is progressively displayed on the tablet. The method continues by detecting a progressive tracing of the displayed visual symbol from the progressive input of the subject user. Next, the method determines a score based upon a characteristic of the progressive tracing, the characteristic of the progressive tracing being at least one of how quickly the progressive tracing follows the progressive display of the visual symbol over time on the tablet and how far a path of the progressive tracing on the tablet deviates from a path of the progressive display of the visual symbol on the tablet over time. Finally, the method stores the determined score as a record on a memory device, where the record is associated with the subject user's eye-hand coordination at a particular instance in time.

Another aspect of the disclosure relates to an apparatus for measuring and quantifying eye-hand coordination of a subject user applicable to a three-dimensional operating environment. The apparatus includes, at least in part, a three-dimensional display device, a processor, at least one sensor, and a memory. The three-dimensional display device provides a three-dimensional view of displayed information to the subject user. The processor is in communication with the three-dimensional display device and a sensor, which is configured to accept three-dimensional progressive input from the subject user. The memory is also in communication with the processor and configured to maintain a plurality of visual cues and a plurality of records associated with measuring the eye-hand coordination of the subject user. The processor is configured to progressively display at least one of the visual cues as a three-dimensional visual symbol on the three-dimensional display device, detect a three-dimensional progressive tracing of the displayed visual symbol from the progressive input of the subject user, determine a new eye-hand coordination score based upon how quickly the three-dimensional progressive tracing follows the progressive three-dimensional display of the visual symbol over time and how far a path of the three-dimensional progressive tracing deviates from a path of the three-dimensional progressive display of the visual symbol over time, and store the new eye-hand coordination score within a new record in the memory.

Additional advantages of the disclosure will be set forth in part in the description which follows, and in part will be obvious from the description, or may be learned by practice of the disclosed exemplary embodiments.

Aside from the structural and procedural arrangements set forth above, the embodiments could include a number of other arrangements, such as those explained hereinafter. It is to be understood that both the foregoing description and the following description are exemplary only.

### BRIEF DESCRIPTION OF THE DRAWINGS

The accompanying drawings, which are incorporated in and constitute a part of this description, illustrate several exemplary embodiments and together with the description, serve to explain principles of the embodiments. In the drawings,

FIGS. 1A-1D are perspective views of an exemplary apparatus for measuring and quantifying eye-hand coordination in accordance with an exemplary embodiment of the present invention;

FIG. 2 is a perspective view of an alternative exemplary apparatus for measuring and quantifying eye-hand coordination in accordance with another exemplary embodiment of the present invention;

FIG. 3 is a block diagram of an exemplary apparatus for measuring and quantifying eye-hand coordination in accordance with an exemplary embodiment of the present invention;

FIGS. 4A-4C are perspective views of an exemplary tablet illustrating a visual symbol being progressively displayed and removed along a progressive display path and a tracing being progressively detected and displayed along a progressive tracing path in accordance with an exemplary embodiment of the present invention;

FIG. 5 is a flow diagram of an exemplary method for measuring and quantifying eye-hand coordination in accordance with an exemplary embodiment of the present invention;

FIG. 6 is a flow diagram of an exemplary method for measuring and quantifying eye-hand coordination in accordance with another exemplary embodiment of the present invention; and

FIG. 7 is a block diagram of an exemplary apparatus for measuring and quantifying eye-hand coordination in three dimensions in accordance with an exemplary embodiment of the present invention.

### DESCRIPTION OF EXEMPLARY EMBODIMENTS

Reference will now be made in detail to exemplary embodiments. Wherever possible, the same reference numbers are used in the drawings and the description to refer to the same or like parts.

In general, embodiments of an apparatus and method for measuring and quantifying eye-hand coordination of a subject user are described herein. One or more visual symbols are progressively displayed on a display surface, such as an interactive surface of a tablet, where a progressive tracing of the displayed symbols can be detected based upon progressive input received from the user. Based upon various characteristics of the tracing, such as its path, accuracy, and/or how quickly the user completes the tracing, a score is determined and stored relative to the specific user. Thus, through repeated performance of following the developing progressive lines of the visual symbol, the subject user's eye-hand coordination may be measured and repeatedly quantified.

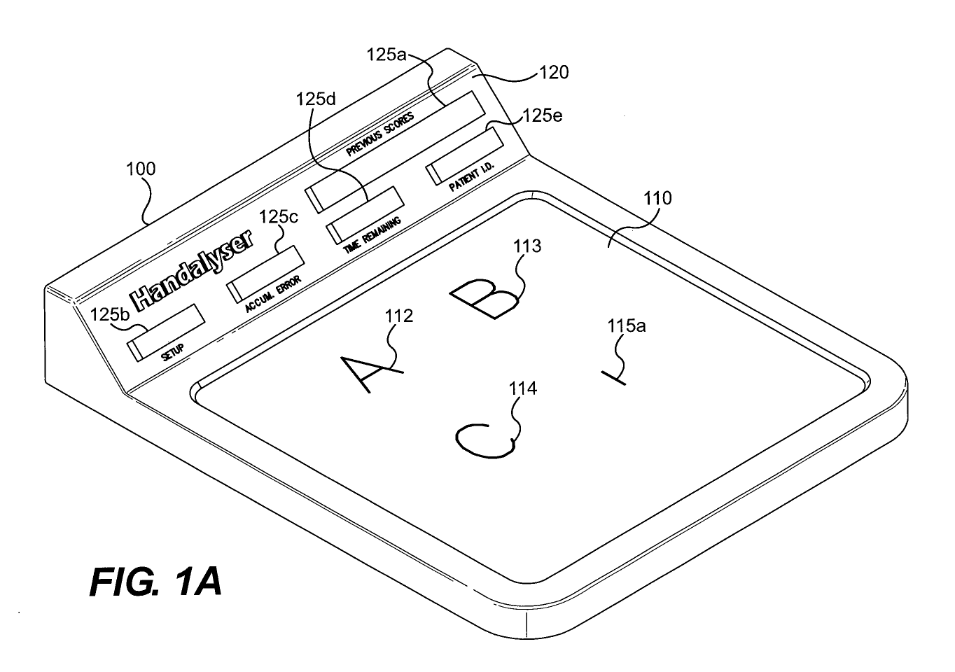

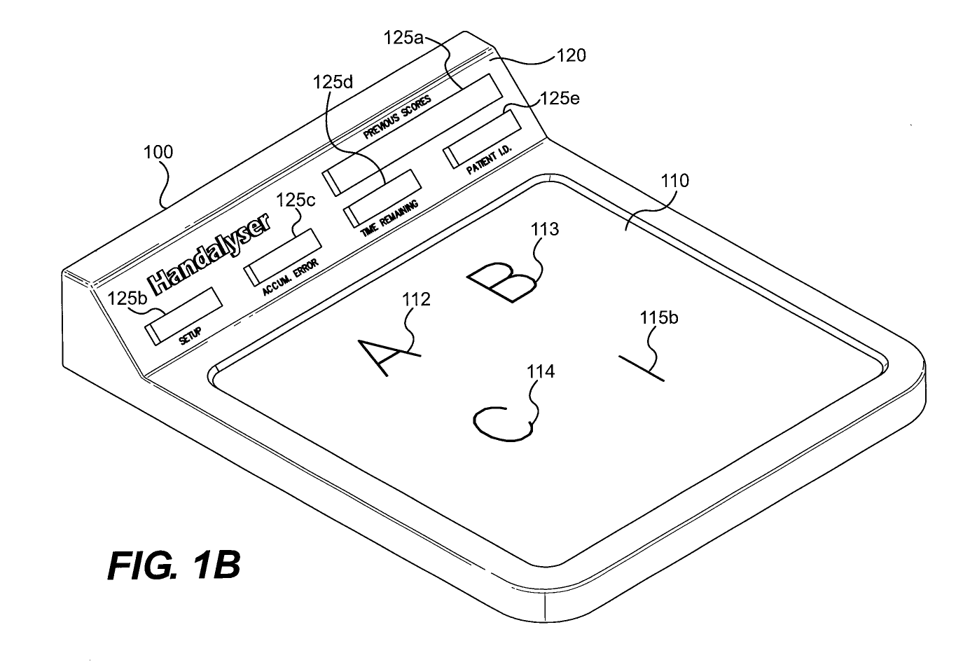

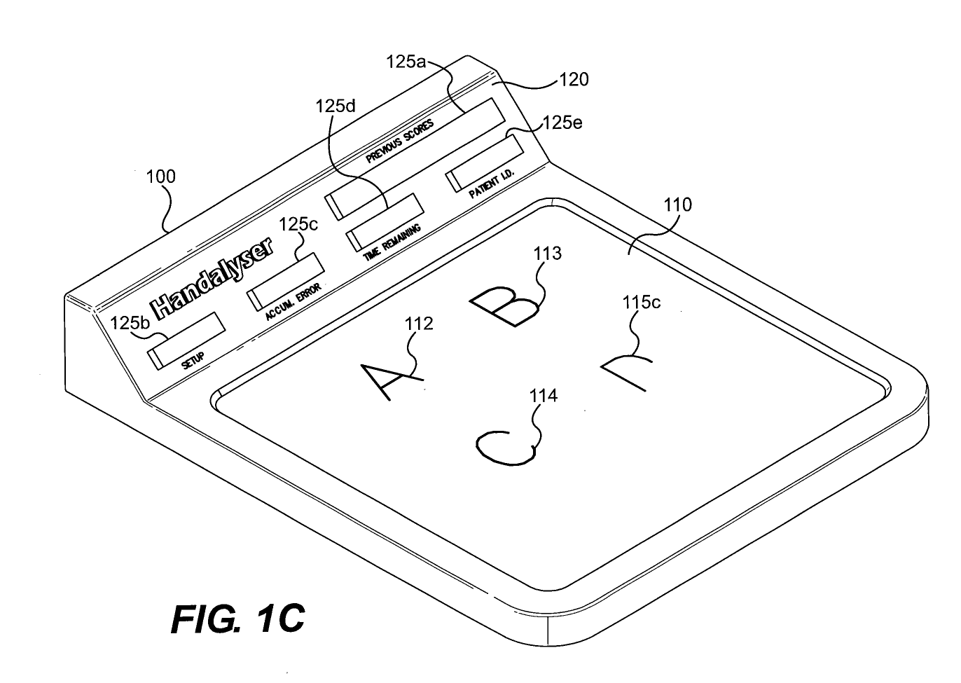

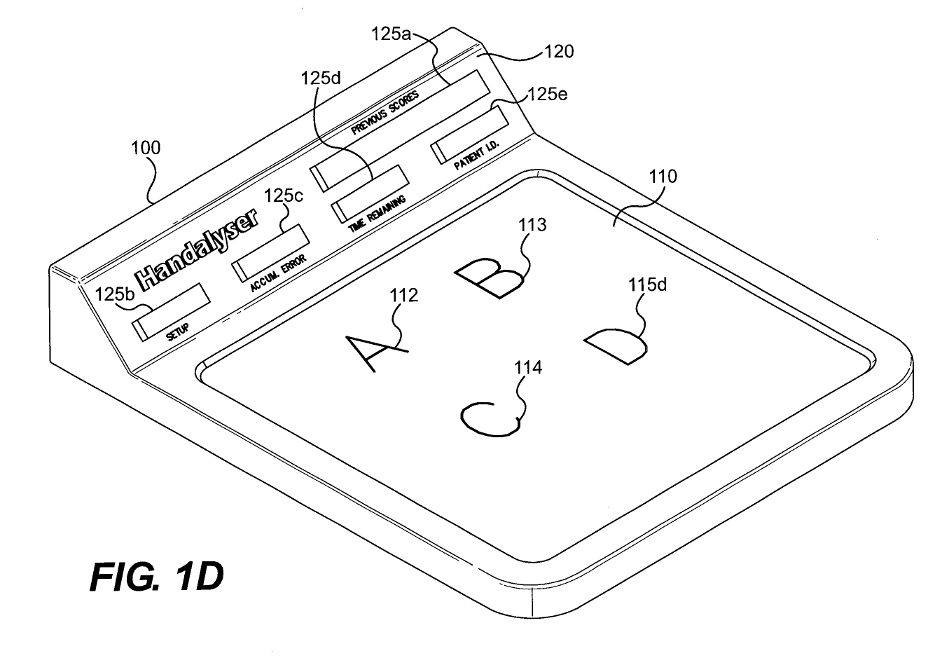

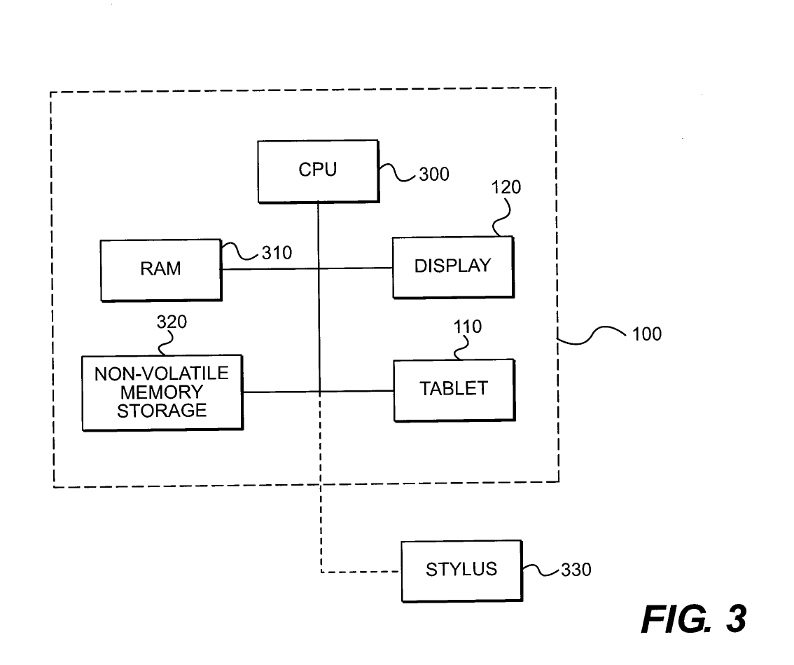

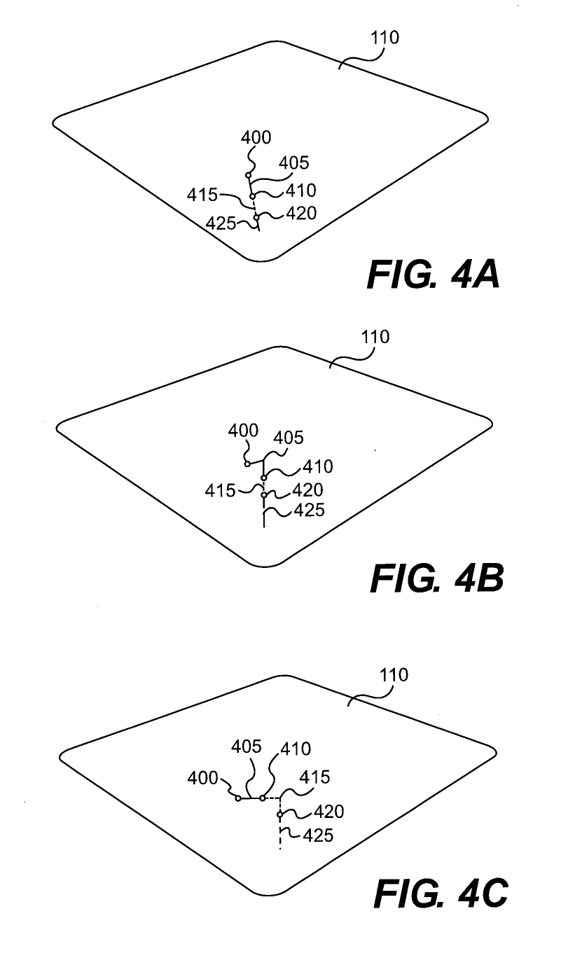

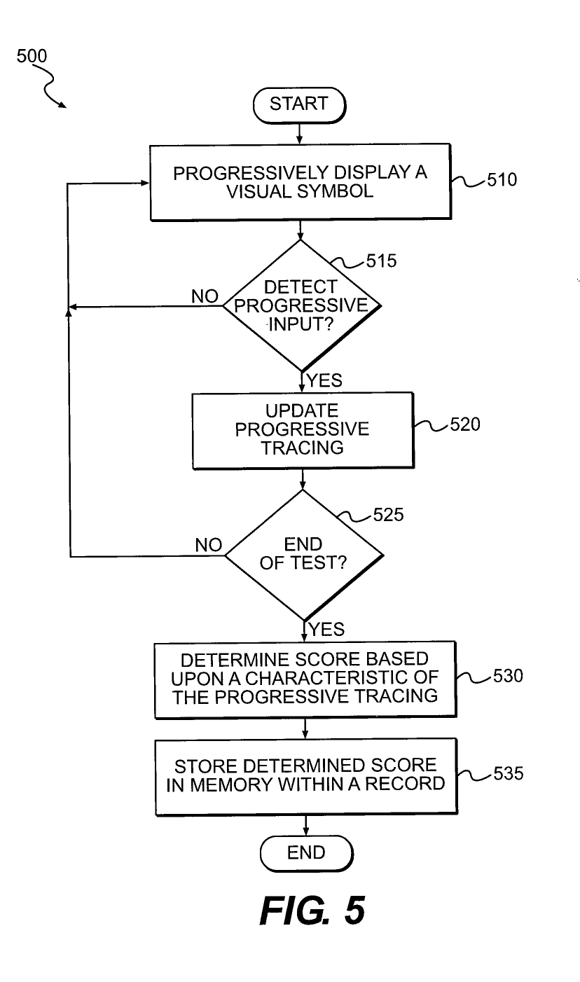

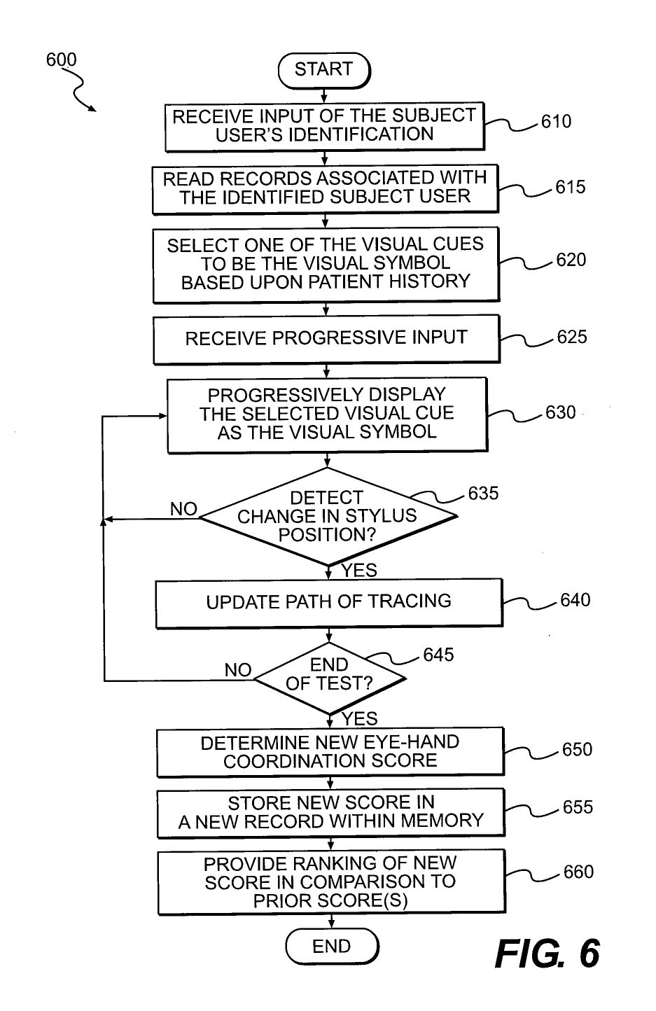

In overview, FIGS. 1A-1D, 2 and 7 are perspective illustrations of different exemplary apparatus, while FIG. 3 provides an general functional block diagram setting forth interrelated operational parts of such exemplary apparatus. FIGS. 4A-4C are illustrative diagrams showing how a visual symbol may be progressively displayed along a first path, with a newer part being displayed and an older part being removed, followed by a detected progressive tracing along another path that follows the appearing and disappearing visual symbol. FIGS. 5 and 6 are flow diagrams providing overviews of exemplary steps performed during operation of exemplary apparatus in accordance with the present invention.

Referring now to FIG. 1A, an exemplary testing unit 100 is shown as a housing that includes a tablet 110 and a display 120 disposed in respective openings of the unit's housing. In general, unit 100 is implemented as a processor-based, touch sensitive and self-contained unit, as shown in FIGS. 1A-1D. Unit 100 is used for measuring and quantifying the eye-hand coordination of a subject user. While not shown in FIGS. 1A-1D, unit 100 incorporates a memory that stores programmatic instructions that, when executed, provide functionality and control of the unit 100. The memory also includes, amongst other things, records with scores and other data related to the eye-hand coordination of a particular subject user.

Those skilled in the art will appreciate that a processor is used herein as a general term for one or more logic devices that are able to control an apparatus with inputs and conditional outputs, including but not limited to combinational logic circuits, general purpose microprocessors, programmable logic devices or programmable logic arrays (PLA). And while exemplary unit 100 is described herein as microprocessor based, other variations of such a unit may be implemented with similar functionality with hard wired circuits or other logic circuits to function without the need for a programmable microprocessor.

Tablet 110 may be implemented as a touch sensitive input device configured to display one or more different possible visual cues as a particular visual symbol, such as symbols 112, 113, 114, and 115a. The tablet 110 receives input from the subject user via touch by detecting the presence, relative location and movement of the user's finger when pressed against a display surface of tablet 110. Alternatively, the tablet 110 may receive input from the user by detecting the presence, relative location and movement of a stylus (not shown) as it is held against the display surface of tablet 110 and moved relative to that surface.

Display 120 of unit 100 is shown as generally having various interfaces, e.g., 125a-125e, that provide useful information to the user. In the illustrated embodiment of FIGS. 1A-D, such interfaces include a previous scores interface display 125a, a setup interface display 125b, an accumulated error interface display 125c, a time remaining interface display 125d, and a patient I.D. interface display 125e. Unit 100 may request the user to enter a patient I.D. through the tablet 110, where the user may enter such information for display on interface 125e. Based upon such patient identification information, the unit 100 looks up and displays previous eye-hand coordination test scores on interface display 125e. The unit 100 is able to provide setup information, such as information on the particular test being run or information needed from the user, on interface display 125b. As the test proceeds and the subject user interacts with unit 100 via tablet 110, unit 100 shows the time remaining for the test on interface display 125d. A score, such as an accumulated error score, may be displayed by unit 100 on interface display 125c. The score may be determined and shown as an ongoing, substantially real-time score or, alternatively, as a score at the end of the test.

In operation, based upon patient identification and the subject user's prior scores, the tablet 110 of unit 100 displays a particular visual symbol to be traced by the subject user once the test begins. Patient identification may be in the form of a user response to a prompt appearing on one of unit 100's displays (include the surface of tablet 110) or, alternatively, in the form of an electronic signal received by the unit 100 from an external source (not shown), such as a remote computer used in a rehabilitation or clinical environment. Visual cues may be of any type of scenes, shapes, objects, numbers or letters, such as the exemplary visual symbols shown in FIGS. 1A-1D. The unit 100 may select which of the possible visual cues to use as displayed visual symbols for a particular user based upon the user's prior scores and history of eye-hand coordination, including an improvement factor for the particular user. Alternatively, the user may select a group of visual cues to use as the visual symbols to be presented to the user.

In the example shown in FIG. 1A, tablet 110 has already progressively displayed the visual symbols A 112, B 113, and C 114 to the subject user and is in the process of progressively displaying the visual symbol D 115a. As shown in FIGS. 1B-1D, the symbol D 115b-115d is progressively displayed on tablet 110. As the symbol is progressively displayed, the subject user attempts to trace the symbol as it progressively appears. In one embodiment, unit 100 provides a score based upon a characteristic of the progressive tracing, such as how quickly or how accurately the user progressively traces the symbol, e.g., 115a-d, as it appears. The unit 100 may also provide a ranking of the user's current score in comparison to at least one prior score as a way to quantify an improvement factor for the user indicating improvement/degradation of eye-hand coordination for the user over time.

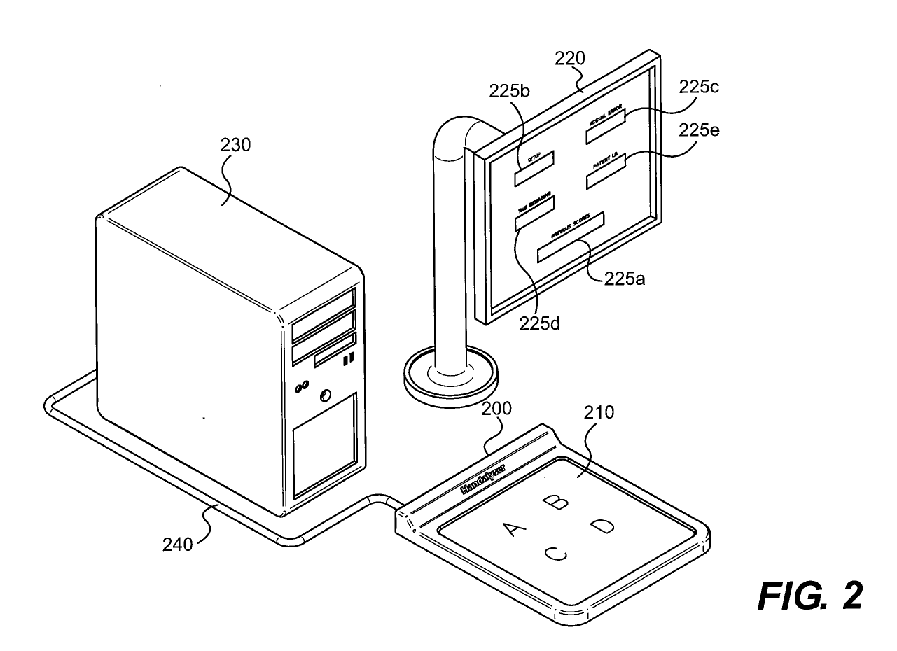

FIG. 2 is a perspective view of an alternative exemplary apparatus for measuring and quantifying eye-hand coordination in accordance with another exemplary embodiment of the present invention. Referring now to FIG. 2, an exemplary testing unit 200 is shown as having separate components, such as a general purpose computer 230, a housing 200 having an interactive tablet 210, and a display 220. Similar functions as described with the embodiment illustrated in FIGS. 1A-1D are achieved with the embodiment illustrated in FIG. 2, but with the components being physically separate rather than combined within a unitary housing, such as the housing of unit 100. For instance, general purpose computer 230 need not be a dedicated processor committed solely for the function of measuring and quantifying a subject user's eye-hand coordination. Instead, the processing unit functionality of the general purpose computer 230 may allow further integration with remote memory storage (not shown) accessible via a data communications network (not shown), such as a local area network or wide area network. Similar display interfaces, e.g., interfaces 225a-225e, may appear on the user interface of display 220 to provide a similar user interface to that described above regarding unitary housing unit 100.

FIG. 3 is a block diagram of an exemplary apparatus for measuring and quantifying eye-hand coordination in accordance with an exemplary embodiment of the present invention. Referring now to FIG. 3, the functional block diagram of unit 100 is shown diagrammatically as including a microprocessor (CPU) 300, RAM 310, non-volatile memory storage 320, each of which are in operative communication with and interfaced to tablet 110 and display 120. As previously mentioned, the tablet 110 may receive input from the user by detecting the presence, relative location and movement of a stylus, such as stylus 330, as it is held against the display surface of tablet 110 and moved relative to that surface. The stylus 330 may be implemented with a simple mechanical device used to more accurately provide a distinct input point on the display tablet 110 when compared to that of the user's finger. Alternatively, the stylus 330 may be implemented as a more intelligent stylus that is electrically connected to unit 100 and the interfaced tablet 110 such that detecting a progressive tracing from progressive input of the subject user is coordinated with the interface between stylus 330 and tablet 110.

CPU 300 is implemented as a microprocessor capable of accessing RAM 310, where program code (not shown) for operating unit 100 resides during operation. The program code is initially stored within memory storage 320 as one or more sets of executable instructions. CPU 300 typically reads the appropriate program code from memory storage 320 upon initialization. From there, CPU 300 runs the program code, which in turn, controls operation of unit 100 as the subject user interacts with the unit 100 as part of measuring and quantifying the user's eye-hand coordination in accordance with embodiments of the present invention. The steps shown in FIGS. 5 and 6 operationally describe exemplary algorithmic steps of such program code operation according to embodiments of the present invention.

Memory storage 320 is implemented within unit 100 as a local memory storage, but alternatively may be implemented as a remote memory storage device accessible by CPU 300 through a data communication network interface (not shown). Thus, embodiments of the present invention may implement memory storage 320 as a variety of computer-readable medium including, but not limited to, a hard disk drive, a floppy disk drive, a flash drive, an optical drive, or small format memory card such as a Secure Digital (SD) card. Thus, other embodiments of the present invention may provide the program code on removable memory storage or on memory storage located remotely from the actual testing unit, such as unit 100 or unit 200. Memory storage 320 also stores and maintains determined scores after a subject user completes a test using unit 100 as well as prior scores for a particular subject user.

In operation, the visual symbol being progressively displayed may be selected from one of multiple possible visual cues (not shown) stored in memory storage 320 or generated from the program code resident on memory storage 320. The visual cues may be stored in memory storage 320 as separate code representing the particular visual symbols to be displayed or, alternatively, may be stored in memory storage 320 as part of the operational program code initially loaded by CPU 300 into RAM 310 described above. Selection of which visual cues to use as part of the visual symbol being progressively displayed may depend upon the patient I.D. information associated with the user, prior scores stored in records of memory storage 320 indicative of past performance by the user on eye-hand coordination tests, or determined rankings of the user's improvement of eye-hand coordination.

Additionally, CPU 300 may cause the tablet 110 to progressively display the visual symbol by progressively removing an older portion of the visual symbol while progressively displaying a newer part of the visual symbol while detecting the progressive tracing of the appearing and disappearing visual symbol. FIGS. 4A-4C are perspective views of an exemplary tablet illustrating how a visual symbol may be progressively displayed and removed along a progressive display path and a tracing that is progressively detected and displayed along a progressive tracing path in accordance with an exemplary embodiment of the present invention.

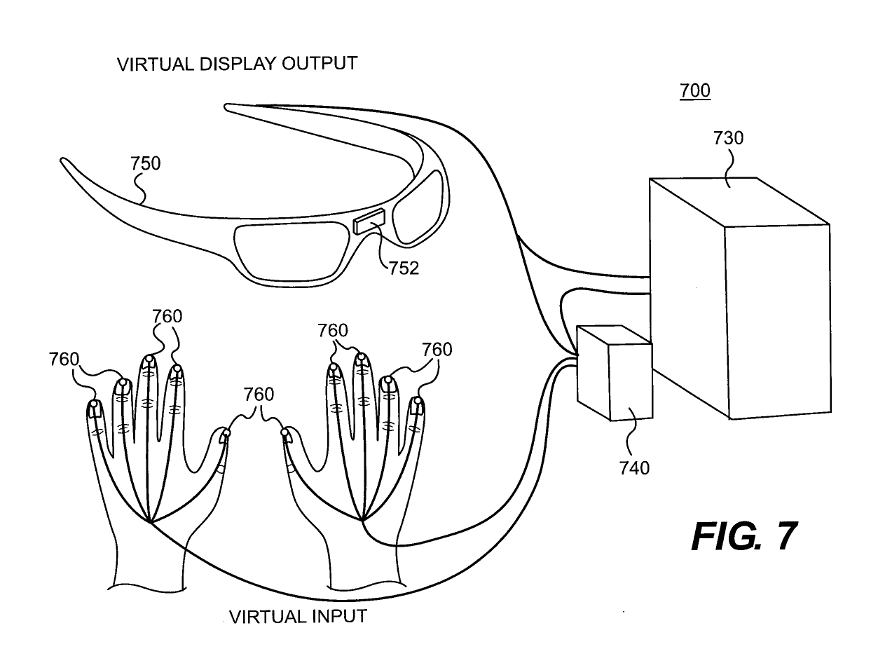

Referring now to FIG. 4A, a display surface of tablet 110 is shown as depicting a visual symbol 405 being progressively displayed. Specifically, a newer portion 400 of the visual symbol 405 is progressively displayed while an older portion 410 of the visual symbol 405 is removed along a path 415 of the progressive display. Following the progressively displayed visual symbol 405, a progressive tracing 425 is detected from progressive input 420 of the user. As the progressively displayed visual symbol 405 appears on tablet 110, the subject user attempts to trace the symbol 405 as shown in FIGS. 4B and 4C where the progressive input 420 moves relative to the visual symbol 405 along a traced path. Those skilled in the art will appreciate that the principles of progressive display and removal of a displayed path or trace are equally applicable in the context of a three-dimensional input and display environment, such as the exemplary embodiment described with reference to FIG. 7.

In general, CPU 300 is configured to determine a score for the subject user upon completing the test based upon one or more characteristics of the detected progressive tracing. For example, in one embodiment, the score is based upon how quickly the user is able to trace the visual symbol over time. In another embodiment, the score may be based upon how accurate the progressive tracing is relative to the path of the progressively displayed visual symbol, such as how far the progressive tracing path deviates from a path of the progressively displayed visual symbol. In some embodiments, CPU 300 is configured to provide feedback and interim test results in the form of accumulated scores. However, other embodiments may configure the CPU 300 to determine the subject user's score at the completion of the test as a final score.

In addition to these general examples and accompanying description for how an embodiment of the present invention may determine a score, several more detailed examples for determining a score in accordance with principles of the present invention are provided with reference to Table 1 below.

TABLE 1

| Example | Description |
|---|---|
| Score 1 | (Multiplier × Distance)/(Speed of Trace) |
| Score 2 | (Multiplier × (Speed of Trace))/(Distance + Constant) |
| Score 3 | Multiplier × (Elapsed Contact Time) × (Contact Position |
| | Error) |
| Score 4 | Multiplier × (Maximum Distance Error for a Visual |
| | Symbol) |
| Score 5 | Multiplier × (Total Accumulated Position Error for a Visual |
| | Symbol) |
| Scores 6-10 | Scores 1-5 with Distance computed from straight line error |
| Scores 11-18 | Scores 3-10 inverted (e.g., Score 11 = Multiplier/(Score |
| | 3 + constant) |

Score 1 may be determined in one embodiment by determining the “Distance” as the length of the traced arc along the path of the progressive display (e.g., path 415 shown in FIGS. 4A-4C and not merely the straight line distance between the display and traced input point), and calculating the product of that Distance and a preselected “Multiplier” value, which is then divided by the speed of the trace. In this embodiment, a perfect score of zero indicates the subject user is tracing the progressively displayed visual symbol as it is being displayed with no lagging distance from where the newer portion 400 of the visual symbol 405 is displayed and the progressive input 420 of the user. Realistically, there is likely to be some minimal lagging distance as the “Distance” for Score 1, but those skilled in the art will appreciate that a lower score of Score 1 is indicative of an increased hand-eye coordination for the subject user.

Score 2 is an inverse of Score 1 with the “Constant” being added to the Distance to prevent divide-by-zero errors. Score 2 may also be determined as a percentage when compared to when the Distance is a zero value (the ideal perfect score). Thus, an implementation of Score 2 as a percentage may be determined as (100)×(Score 2)/(Score 2 when Distance is a zero value).

Score 3 is a type of score that may be helpful in gauging initial reaction time between observation with the eye and coordination with hand movements. The Mulitplier value is multiplied times two different factors: (1) the time between the first appearance of a particular visual symbol and when the subject user first provides the initial point of progressive input for tracing that visual symbol (i.e., the Elapsed Contact Time), and (2) the position error between the first appearance of the particular visual symbol and the initial point of progressive input from the subject user when attempting to trace that visual symbol (i.e., Contact Position Error).

Score 4 is the product of the Multiplier and a maximum value of the Distance determined when the subject user is attempting to trace a particular visual symbol. Likewise, Score 5 is the product of the Multiplier and a sum of the Distances incrementally determined over time as the subject user is attempting to trace a particular visual symbol. Thus, Score 4 is a maximum error type of scoring measurement while Score 5 is an accumulated error type of scoring measurement.

Another example of scoring alternatively determines the Distance as a pure linear distance between points (e.g., the straight line distance between the newest point displayed on the progressively displayed visual image and the latest progressive input point when the subject user attempts to trace the path of the progressively displayed visual symbol) as opposed to the distance computed along the progressively displayed path (which may be different than the straight line distance). Scores 6-10 are types of scores determined in accordance with the exemplary Scores 1-5, but with the Distance value determined as a straight line Distance. Depending on the configuration of the visual symbol being progressively displayed, using a straight line Distance may be less taxing on the unit to compute.

As with Scores 1 and 2, which have an inverted relationship, other scores may be used that are inverted versions of such exemplary scores. For example, Scores 11-18 correspond to the factors and calculations used to determine Scores 3-10, respectively, but are merely inverted.

In some embodiments, Scores 1 and/or Score 2 may be determined and displayed in substantially real time as instantaneous scores. In other embodiments, they may also or alternatively be determined as averages and, at the end of the test, the last average may be used as the respective final score for the test. Embodiments of the invention may also or alternatively determine Scores 3, 4 and/or 5 at the end of each progressively displayed visual symbol and, as such, Scores 3, 4 and/or 5 would be updated incrementally rather than in a continuous or near real time manner. However, it is anticipated that embodiments of the present invention implementing Scores 3, 4, and 5 may determine these scores as averages and, at the end of the test, the last average may be used as the final score for the test.

Further details of the operation and functionality of embodiments of the present invention may be understood with reference to the flow diagrams set forth in FIGS. 5 and 6. FIG. 5 is a general flow diagram of an exemplary method for measuring and quantifying eye-hand coordination in accordance with an exemplary embodiment of the present invention. The method 500 begins by progressively displaying a visual symbol at stage 510. In some embodiments, the visual symbol may have an older portion that is progressively removed while a newer portion of the visual symbol is progressively displayed. At stage 515, a determination is made if any progressive input from the user is detected. If so, stage 515 proceeds to stage 520. If not, stage 515 proceeds back to state 510 where the visual symbol continues to be progressively displayed.

At stage 520, method 500 updates the progressive tracing from the detected progressive input from the user before proceeding to stage 525. At stage 525, if the time for the test is at an end, stage 525 proceeds to 530 for scoring. However, if the test is not yet ended, stage 525 proceeds back to stage 510 where the visual symbol continues to be progressively displayed and stages 515 and 520 where progressive input is received and the progressive tracing is continued to be detected.

At stage 530, the method 500 determines a score based upon a characteristic of the progressive tracing. In one embodiment, the score may be based upon how quickly the progressive tracing follows the progressive display of the visual symbol over time. In another embodiment, the score may be based upon how far a path of the progressive tracing deviates from a path of the progressive display of the visual symbol over time. Such deviations may be computed as an average accumulated error distance of the tracing off the path of the progressively displayed visual symbol. In some embodiments, scoring may be determined on an ongoing, accumulated basis instead of just at the completion of the test, and as such, may be periodically shown to the user during the test.

After the score is determined at stage 530, the determined score is stored within a record in memory at stage 535 before method 500 ends. The record may be of any predetermined format or data structure within volatile memory, such as RAM 310, or non-volatile memory, such as memory storage 320. The record may include the determined score at the end of the test or may also include details of the test (e.g., ongoing accumulated scores during the time, a time profile of such periodic accumulated scores, information relating to the tracing deviations and/or time lag information relating to how quickly the subject user was tracing the visual symbol, etc.).

FIG. 6 is a flow diagram of another exemplary method for measuring and quantifying eye-hand coordination in accordance with an exemplary embodiment of the present invention. Referring now to FIG. 6, method 600 begins by receiving input of the subject user's identification at stage 610. In some embodiments, the input may be in the form of a user response directly on tablet 110. In other embodiments, the input may be in the form of an electronic signal received or electronic information read from a memory storage where the electronic signal/information stored reflects information associated with the subject user's identity (e.g., a patient number, name, etc.). At stage 615, method 600 reads the records associated with the identified subject user. At stage 620, method 600 selects one of the visual cues (or sets of visual cues) to be the visual symbol (or set of visual symbols) that will be displayed to the subject user during the test based upon the subject user's patient history. For example, the patient's history may reflect a particular progression of tests having been completed for certain visual cues or sets of visual cues according to a predetermined protocol of treatment and testing.

At stages 625 and 630, method 600 receives progressive input from the subject user while progressively displaying a visual symbol from the selected visual cue(s). In some embodiments, the visual symbol may have an older portion that is progressively removed while a newer portion of the visual symbol is progressively displayed. At stage 635, a determination is made if change in stylus position is detected as updated progressive input from the subject user. If so, stage 635 proceeds to stage 640. If not, stage 635 proceeds back to state 630 where the visual symbol continues to be progressively displayed.

At stage 640, the path of tracing is updated. In some embodiments, the location information of the user's latest trace input may be recorded with reference to elapsed time. At stage 645, if the test had ended, method 600 proceeds to stage 650 for scoring. However, if the test has not yet ended, method 600 proceeds back to stage 630 where the visual symbol continues to be progressively displayed.

At stage 650, method determines a new eye-hand coordination score based upon a characteristic of the progressive tracing. In one embodiment, the score may be based upon how quickly the progressive tracing follows the progressive display of the visual symbol over time. In another embodiment, the score may be based upon how far a path of the progressive tracing deviates from a path of the progressive display of the visual symbol over time. Such deviations may be computed as an average accumulated error distance of the tracing off the path of the progressively displayed visual symbol. In some embodiments, scoring may be determined on an ongoing, accumulated basis instead of just at the completion of the test, and as such, may be periodically shown to the user during the test and maintained for later storage in memory associated with the current test.

After the score is determined at stage 650, the determined new score is stored within a new record in memory at stage 655. As mentioned previously, such a new record may be of any predetermined format or data structure within volatile memory, such as RAM 310, or non-volatile memory, such as memory storage 320. The record may include the determined score at the end of the test or may also include details of the test (e.g., ongoing accumulated scores during the time, a time profile of such periodic accumulated scores, information relating to the tracing deviations and/or time lag information relating to how quickly the subject user was tracing the visual symbol, etc.).

At stage 660, method 600 also may determine and provide a ranking of the new score in comparison to one or more prior scores for the subject user. Alternatively, the ranking may be in comparison to other standards or statistics other than the subject user's actual prior scores, such as general population statistical information relating to eye-hand coordination. Such rankings may provide an indication of progress for prescribed therapy that is intended to address and improve the subject user's eye-hand coordination skills.

While the principles of the present invention as exemplified through the embodiments described above for measuring and quantifying eye-hand coordination rely upon two-dimensional (2D) input and output, those skilled in the art will appreciate that alternative embodiments of the present invention may be implemented with three-dimensional (3D) input and output. Generally, an exemplary embodiment of the present invention may progressively display and remove the path and trace of a visual symbol in three-dimensions (e.g., via output seen through a three-dimensional head set, goggles or other vision system) and detect three-dimensional input from the subject user (e.g., via input from a user-manipulated three-dimensional input device, such as sensors in a hand glove).

FIG. 7 is a block diagram of an exemplary apparatus for measuring and quantifying eye-hand coordination implemented with the capability to receive 3D input from the subject user and outputting information to the subject user in a 3D manner in accordance with an exemplary embodiment of the present invention. Referring now to FIG. 7, an exemplary testing unit 700 is shown as having separate components, such as a general purpose computer 730, a tracker system 740, a display device 750, and one or more sensors 760. These components are coupled to and in operative communication with each other. The general purpose computer 730 may be similar to computer 230 used in a 2D embodiment with additional software and interfaces, as needed, to communicate with the tracker system 740, display device 750 and input sensors 760, so as to detect, process and provide information regarding 3D user input, 3D progressive displayed paths, 3D progressive traces, and other ongoing or scoring information to the user in a 3D manner.

In one embodiment, the tracker system 740 is generally implemented as one or more communication ports (such as a universal system bus (USB), serial, parallel, or other data communication interface) that links the computer and sensor/display device. The tracker system 740 provides access by computer 730 to positional signals generated by one or more input sensors 760 and the display device 750 while providing signals from the computer 730 to the display device 750 as a 3D user interface. In one embodiment, the tracker system 740 provides a wired interface between computer 750 and the sensors/display device. In other embodiments, the tracker system 740 may use a wireless transmitter and receiver in each of the respective devices to facilitate provision of signals from the computer 730 to each of the sensors 760 and display device 750 and reception by the computer 730 of signals generated from each of the sensors 760 and display device 750.

The 3D embodiment of the present invention illustrated in FIG. 7 allows the user to view a progressively displayed visual symbol in 3D via display device 750, such as a stereoscopic set of virtual reality goggles or other three-dimensional display systems where a user is presented with an item in what appears to be a three-dimensional virtual reality or projected 3D image against an otherwise real backdrop. Likewise, the embodiment shown in FIG. 7 allows the subject user to move sensors 760, such as spatially oriented sensors attached to a user-manipulated glove (as shown in FIG. 7) or a user-manipulated stylus (not shown) that provides a 3D point of reference in space. Signals from the sensors provide a 3D coordinate position of the sensors relative to a reference point. Likewise, a signal from the display device, e.g., from transmitter 752, provides a 3D position of the display device relative to the same reference point. In this manner, the sensors 760 and display device 750 are coordinated, via the tracker system 740, by application software running in memory on computer 730. The computer 730 is operative to detect points in three-dimensional space as detected input from the subject user and display three-dimensional representations of paths in a display space (e.g., progressively displayed and removed visual symbols, progressively displayed and removed paths of a subject user's attempt to trace the progressively displayed visual symbols).

In the context of such three-dimensional input and display output for the subject user, principles of the present invention, the application software running on computer 730 may implement the exemplary methods described with respect to FIGS. 5 and 6. Additionally, applying the general principles of the present invention to a three-dimensional embodiment may also have the advantage of measuring and quantifying eye-hand coordination at a more complex or otherwise different level when compared with a two-dimensional embodiment. Testing, tracking, scoring and ranking of a subject user's ability to trace a progressively appearing visual symbol in three-dimensions may have also have further utility beyond that of therapy (e.g., training assessment, etc.).

Those skilled in the art will appreciate that the details of a computer or processor-based apparatus and computer-implemented method for receiving three-dimensional user input and displaying three-dimensional output are well known. Such well known apparatus may provide the operating platform for embodiments of the present invention. For example, details of an implementation for such an apparatus consistent with the embodiment described in FIG. 7 is disclosed in more detail in U.S. Patent Application Publication No. 2005/0264527 A1, which is hereby incorporated by reference.

Principles of the present invention, which include measuring and qualifying eye-hand coordination of a subject user, may be applied with embodiments used in a teaching environment. For example, embodiments of the present invention may be used to help teach a subject user how to draw, sketch or paint in two dimensions (e.g., via a tablet interface) or in three-dimensions (e.g., via the 3D input and output devices referenced in FIG. 7). In an exemplary teaching embodiment, the visual symbol to be traced or followed by the subject user may represent a 2D or 3D image of an object to be replicated by the subject user in a simple monochromatic fashion or in a multi-colored fashion.

In more detail, an embodiment of the present invention may be used to teach a subject user how to sketch or draw with a guided measurement and quantification of eye-hand coordination as set forth relative above. Relevant embodiments used to measure and quantify eye-hand coordination and teach drawing may have a memory that maintains files associated with one or more composite images to be drawn. Each file for a composite image may include one or more visual items. The visual items collectively make up the composite image to be drawn by the subject user. For example, one file may include four distinct visual items that collectively make up a composite image of a person's face (e.g., two eyes, a nose and a mouth).

In operation of such an embodiment, the processor in the apparatus is configured, via programmatic instructions, to follow steps outlined generally and described with respect to FIGS. 5 and 6 as each visual item is progressively displayed to the subject user as a visual symbol. As the visual symbol is progressively traced by the user and the user completes tracing the visual symbol, another of the visual items making up the composite image may be progressively displayed. In this manner, different visual symbols are progressively shown to the subject user, who manipulates an input device (e.g., a stylus and tablet, a hand glove with positional sensors) that provides progressive input. The user's tracing is then progressively displayed and scored. Scoring, as described above, may be determined based upon how quickly the subject user's progressive tracing follows the progressive display of the visual symbol over time and how close the tracing comes to the displayed visual symbol (generally how far a path of the tracing deviates from a path of the displayed visual symbol). As such, scoring in this application is associated with a level of drawing skill, which may advantageously improve with time and practice using an embodiment of the present invention.

Another embodiment of the present invention may be used to teach a subject user how to paint as it measures and quantifies eye-hand coordination. In this embodiment, the memory maintains files associated with one or more composite images to be painted. Each file for a composite image may include one or more visual items and related color information assigned to the whole or parts of each visual item. The visual items, including their respective individually colored parts, collectively make up the composite image to be painted by the subject user.

In operation of such an embodiment, the user may be prompted to select a color from a predetermined palette to attempt to match the color associated with a particular visual symbol or part thereof. The visual symbol, or the part of the symbol, is then progressively traced by the user using the selected color. After the user completes painting of the visual symbol, including all of it's individual parts, another of the visual items making up the composite image may be progressively displayed. In this manner, different visual symbols are progressively shown to the subject user, who manipulates an input device (e.g., a stylus and tablet, a hand glove with positional sensors) that provides progressive input to represent painting of the composite image. The user's tracing, which includes outlining and filling of individual parts of the visual symbols, is then progressively displayed and scored. Scoring, as described above, may be determined based upon how quickly the subject user's progressive tracing follows the progressive display of the visual symbol over time and how close the tracing comes to the displayed visual symbol (generally how far a path of the tracing deviates from a path of the displayed visual symbol). Additionally, scoring may include a matching determination of the user's selected color and the visual symbol's assigned color (or it's individual part's color). As such, scoring in this application is associated with a level of painting skill, which may advantageously improve with time and practice using an embodiment of the present invention.

Similar to selection of color, other embodiments may also include selection of other reproduction characteristics (e.g., painting characteristics, drawing characteristics). For example, an embodiment may have the system or apparatus prompt selection of a pattern (e.g., dots, streaks, etc.), representative brush type and shape, (e.g., round, flat, fan, angle, Filbert, etc.), and texture to be applied (oil-like thick appearance, watercolor-like smooth appearance, charcoal-like rough appearance, etc). In such embodiments, the file for the composite image would maintain predetermined stored characteristics for each visual item's assigned painting characteristics (e.g., pattern, correct brush type to be used when painting, and texture to be applied). As the visual symbol is progressively displayed, the progressive input from the user is shown on the relevant display as corresponding to painting with the selected painting characteristics.

Although aspects of the exemplary embodiments disclosed herein are explained in relation to a specific computer or microprocessor based system with programmatic instructions, it should be understood that the exemplary embodiments described herein could be used in systems with processors that are hard wired with programmatic instructions for providing the novel functionality and operations.

Thus, at least some portions of exemplary embodiments of the systems outlined above may used in association with portions of other exemplary embodiments. Moreover, at least some of the exemplary embodiments disclosed herein may be used independently from one another and/or in combination with one another and may have applications to devices and methods not disclosed herein.

It will be apparent to those skilled in the art that various modifications and variations can be made to the structures and methodologies described herein. Thus, it should be understood that the invention is not limited to the subject matter discussed in the description. Rather, the present invention is intended to cover modifications and variations.

What is claimed is:

1. An apparatus for measuring and quantifying eye-hand coordination of a subject user, the apparatus comprising:
a processor;
a tablet interfaced with the processor and configured to accept progressive input from the subject user; and
a memory interfaced with the processor and configured to maintain a record associated with measuring the eye-hand coordination of the subject user;
wherein the processor is configured to
progressively display a visual symbol on the tablet,
detect a progressive tracing of the displayed visual symbol from the progressive input of the subject user,
determine a score based upon a characteristic of the progressive tracing, and
store the determined score within the record in the memory.

2. The apparatus of claim 1 wherein the tablet further comprises a touch-sensitive surface.

3. The apparatus of claim 1 further comprising a stylus operated by the subject user and wherein the tablet is configured to detect the presence of the stylus over time to be the progressive input of the subject user.

4. The apparatus of claim 1 further comprising a housing for the processor and the memory and wherein the tablet is disposed external to the housing.

5. The apparatus of claim 1, wherein the processor is further configured to progressively remove an older portion of the visual symbol while progressively displaying a newer portion of the visual symbol, and wherein the processor is further configured to detect the progressive tracing from the progressive input as the visual symbol is progressively displayed and a removed.

6. The apparatus of claim 1, wherein the characteristic of the progressive tracing is how quickly the progressive tracing follows the progressive display of the visual symbol over time.

7. The apparatus of claim 1, wherein the characteristic of the progressive tracing is how far a path of the progressive tracing deviates from a path of the progressive display of the visual symbol over time.

8. The apparatus of claim 1, wherein the processor is further configured to select one of a plurality of visual cues as the visual symbol to be progressively displayed for the subject user based upon a previously determined score for the subject user.

9. The apparatus of claim 1, wherein the processor is further configured to provide a ranking of the determined score for the subject user in comparison to at least one prior determined score for the subject user so as to quantify an improvement factor of the eye-hand coordination of the subject user over time.

10. An apparatus for measuring and quantifying eye-hand coordination of a subject user, the apparatus comprising:
a housing having a first display opening and a second display opening;
a processor disposed within the housing;
a tablet in communication with the processor, the tablet being disposed within the housing and having a display surface oriented for viewing through the first display opening of the housing, the tablet being configured to accept progressive input from the subject user;
a stylus operated by the subject user, wherein the tablet is configured to detect the presence of the stylus as it moved by the subject user relative to the display surface of the tablet over time as the progressive input of the subject user;
a measurement result interface in communication with the processor, the measurement result interface being disposed within the housing and being viewable through the second display opening of the housing; and
a memory in communication with the processor and configured to maintain a plurality of visual cues and a plurality of records associated with measuring the eye-hand coordination of the subject user;
wherein the processor is configured to
select one of a plurality of visual cues stored in the memory as a visual symbol to be progressively displayed for the subject user based upon an analysis of previously determined eye-hand coordination scores for the subject user stored within the records in the memory,
progressively display the selected visual symbol on the tablet,
detect a progressive tracing of the displayed visual symbol from the progressive input of the subject user,
determine a new eye-hand coordination score based upon how quickly the progressive tracing follows the progressive display of the visual symbol over time and how far a path of the progressive tracing deviates from a path of the progressive display of the visual symbol over time, and
store the new eye-hand coordination score within a new record in the memory.

11. The apparatus of claim 10, wherein the processor is further configured to progressively remove an older portion of the visual symbol while progressively displaying a newer portion of the visual symbol, and wherein the processor is further configured to detect the progressive tracing from the progressive input as the visual symbol is progressively displayed and a removed.

12. The apparatus of claim 1, wherein the processor is further configured to provide a ranking on measurement result interface of the new eye-hand coordination score for the subject user in comparison to at least one prior score for the subject user stored within the records in the memory so as to quantify an improvement factor of the eye-hand coordination of the subject user over time.

13. A method for measuring and quantifying eye-hand coordination of a subject user, the method comprising the steps of:
progressively displaying a visual symbol on a tablet;
accepting progressive input from a stylus operated by the subject user as a visual symbol is progressively displayed on the tablet;
detecting a progressive tracing of the displayed visual symbol from the progressive input of the subject user;
determining a score based upon a characteristic of the progressive tracing, the characteristic of the progressive tracing being at least one of how quickly the progressive tracing follows the progressive display of the visual symbol over time on the tablet and how far a path of the progressive tracing on the tablet deviates from a path of the progressive display of the visual symbol on the tablet over time; and
storing the determined score as a record on a memory device, the record being associated with the subject user's eye-hand coordination at a particular instance in time.

14. The method of claim 13, wherein the step of progressively displaying further comprises progressively removing an older portion of the visual symbol from the tablet while progressively displaying a newer part of the visual symbol on the tablet; and
wherein the step of detecting the progressive tracing further comprises detecting the progressive tracing from the progressive input as the visual symbol is progressively displayed and removed on the tablet.

15. The method of claim 14 further comprising the step of timing the difference how quickly the progressive tracing follows the progressive display of the visual symbol and using the difference time when determining the score.

16. The method of claim 15 further comprising the step of determining a distance of how far behind the current progressive input of the subject user lags the newer part of the visual symbol progressively displayed and using the determined distanced when determining the score.

17. The method of claim 14, wherein a speed at which the visual symbol is progressively displayed and removed on the tablet is adjustable based upon the last determined score stored on the memory device and associated with the subject user.

18. The method of claim 13 further comprising the step of selecting one of a plurality of visual cues as the visual symbol to be progressively displayed for the subject user based upon a previously determined score for the subject user.

19. The method of claim 13 further providing a step of quantifying an improvement factor of the eye-hand coordination of the subject user over time by providing a ranking of the determined score for the subject user relative to at least one prior determined score for the subject user.

20. A computer readable medium on which is stored executable instructions for measuring and quantifying eye-hand coordination of a subject user, the instructions when executed comprising the steps of:
progressively displaying a visual symbol on a tablet;
accepting progressive input from a stylus operated by the subject user as a visual symbol is progressively displayed on the tablet;
detecting a progressive tracing of the displayed visual symbol from the progressive input of the subject user;
determining a score based upon a characteristic of the progressive tracing, the characteristic of the progressive tracing being at least one of how quickly the progressive tracing follows the progressive display of the visual symbol over time on the tablet and how far a path of the progressive tracing on the tablet deviates from a path of the progressive display of the visual symbol on the tablet over time; and
storing the determined score as a record on a memory device, the record being associated with the subject user's eye-hand coordination at a particular instance in time.

21. An apparatus for measuring and quantifying eye-hand coordination of a subject user, the apparatus comprising:
a three-dimensional display device that provides a three-dimensional view of displayed information to the subject user;
a processor in communication with the three-dimensional display device;
a sensor in communication with the processor, the sensor being configured to accept three-dimensional progressive input from the subject user;
a memory in communication with the processor and configured to maintain a plurality of visual cues and a plurality of records associated with measuring the eye-hand coordination of the subject user;
wherein the processor is configured to
progressively display at least one of the visual cues as a three-dimensional visual symbol on the three-dimensional display device,
detect a three-dimensional progressive tracing of the displayed visual symbol from the progressive input of the subject user,
determine a new eye-hand coordination score based upon how quickly the three-dimensional progressive tracing follows the progressive three-dimensional display of the visual symbol over time and how far a path of the three-dimensional progressive tracing deviates from a path of the three-dimensional progressive display of the visual symbol over time, and
store the new eye-hand coordination score within a new record in the memory.

22. A method for measuring and quantifying eye-hand coordination of a subject user, the method comprising the steps of:
selecting one or more reproduction characteristics;
progressively displaying a visual symbol on a tablet;
accepting progressive input from a stylus operated by the subject user as a visual symbol is progressively displayed on the tablet;
detecting a progressive tracing of the displayed visual symbol from the progressive input of the subject user;
displaying the progressive tracing on a display device in accordance with the selected reproduction characteristic;
determining a score based upon at least one of
a matching association of the selected reproduction characteristic with a predetermined reproduction characteristic for the visual symbol,
how quickly the progressive tracing follows the progressive display of the visual symbol over time on the tablet, and
how far a path of the progressive tracing on the tablet deviates from a path of the progressive display of the visual symbol on the tablet over time; and
storing the determined score as a record on a memory device, the record being associated with the subject user's eye-hand coordination at a particular instance in time.
